(19) United States
(12) Patent Application Publication
(10) Pub. No.: US 2025/0353905 A1
(43) Pub. Date: Nov. 20, 2025

(54) COMPOSITIONS AND METHODS FOR TREATING POSTURAL TACHYCARDIA SYNDROME

(71) Applicant: HELICORE BIOPHARMA, INC., San Mateo, CA (US)

(72) Inventors: Nir Barak, San Mateo, CA (US); Sireesh Appajosyula, San Mateo, CA (US); Patrick H. Griffin, San Mateo, CA (US)

(21) Appl. No.: 18/872,620

(22) PCT Filed: Jun. 5, 2023
(86) PCT No.: PCT/US2023/067910

§ 371 Date: Dec. 6, 2024

Related U.S. Application Data
(60) Provisional application No. 63/349,630, filed on Jun. 7, 2022.

(51) Int. Cl. C07K 16/26; A61K 39/00

(52) U.S. Cl. CPC C07K 16/26; A61K 2039/54; A61K 2039/545; C07K 2317/24; C07K 2317/76

(57) ABSTRACT

The present invention provides compositions and methods for treating Postural orthostatic tachycardia syndrome (POTS). In some embodiments, the POTS is associated with hypermobility spectrum disorders/hypermobile Ehlers-Danlos syndrome (HSD/hEDS). In various embodiments, the invention relates to administering a composition comprising an effective amount of a molecular antagonist of Glucose-dependent Insulinotropic Polpeptide (GIP) to a patient in need thereof. In various embodiments, the molecular antagonist can be provided in a form for convenient self-administration upon the onset of symptoms or to prevent or reduce postprandial POTS symptoms, or in other embodiments, is administered at a set frequency.

### CROSS-REFERENCE TO RELATED APPLICATIONS

This application claims the benefit of U.S. Provisional Application No. 63/349,630, filed on Jun. 7, 2022, the entire contents of which are hereby incorporated in their entirety.

### FIELD OF THE INVENTION

The present disclosure provides compositions and methods for treating postural orthostatic tachycardia syndrome (POTS) and conditions and symptoms associated therewith.

### DESCRIPTION OF THE TEXT FILE SUBMITTED ELECTRONICALLY

This application contains a Sequence Listing in XML format submitted electronically herewith via Patent Center. The contents of the XML copy, created on May 30, 2023, is named “NMT-039PC_116031-5039.xml” and is 21,646 bytes in size. The Sequence Listing is incorporated herein by reference in its entirety.

### BACKGROUND

Postural orthostatic tachycardia syndrome (POTS) refers to a condition that affects blood flow and impacts about 3 million young men and women in the United States. POTS is a form of dysautonomia—a disorder of the autonomic nervous system that controls and regulates vital bodily functions. POTS is characterized by various symptoms, including lightheadedness, faintness, and rapid increase in heartbeat upon standing up. POTS can also be associated with various gastrointestinal symptoms, and carbohydrate-rich meals can exacerbate symptoms. While POTS is sometimes treated or controlled with medications such as salt tablets, fludrocortisone, pyridostigmine, midodrine, and/or beta-blockers, disease management tools otherwise include thigh-high compression stockings and close management of diet and health.

There is a need for pharmaceutical compositions and methods for treating POTS and associated conditions, including treatments that reduce or prevent symptoms of the condition.

### DETAILED DESCRIPTION

The present invention provides compositions and methods for treating postural orthostatic tachycardia syndrome (POTS), including mild, moderate, or severe POTS. In some embodiments, the POTS is associated with hypermobility spectrum disorders/hypermobile Ehlers-Danlos syndrome (HSD/hEDS). In various embodiments, the invention relates to administering a composition comprising an effective amount of a molecular antagonist of Glucose-dependent Insulinotropic Polypeptide (GIP) to a patient in need thereof. In various embodiments, the molecular antagonist can be provided in a form for convenient self-administration upon the onset of symptoms or to prevent or reduce postprandial POTS symptoms, or in other embodiments, is provided for administration at a set frequency (e.g., about once daily or once weekly). Further, in some embodiments, the compositions described herein reduce presyncopal symptoms associated with POTS.

In accordance with this disclosure, compositions comprising a molecular antagonist of GIP are administered to prevent or reduce symptoms associated with POTS. For example, in some embodiments the compositions and methods reduce chronic presyncopal symptoms associated with POTS, such as those selected from lightheadedness, upright tachycardia, mental clouding, blurred vision, shortness of breath, rapid heartbeat, tremulousness, chest discomfort, headache, nausea, and dizziness. In some embodiments, administration of the composition can result in modulation of and/or increased upright stroke volume and/or reduced upright norepinephrine and/or epinephrine levels and/or modulation of the splanchnic circulation. In some embodiments, administration or use of compositions described herein can result in modulation of and/or decreased plasma levels of GIP.

POTS is a blood circulation disorder characterized by two factors: (1) a specific group of symptoms that frequently occur when standing upright; and (2) a heart rate increase from horizontal to standing of at least 30 beats per minute in adults, or at least 40 beats per minute in adolescents, measured during the first 10 minutes of standing. Symptoms of POTS include, but are not limited to, lightheadedness, upright tachycardia, mental clouding, blurred vision, shortness of breath, rapid heartbeat, tremulousness, chest discomfort, headache, nausea, and dizziness.

In people with POTS, the blood vessels don't respond efficiently to signals to constrict. As a result, the longer an individual stands upright, the more blood pools in the lower half of the body. This leads to insufficient blood returning to, for example, the brain, manifesting as lightheadedness (faintness), brain fog, and/or fatigue. As the nervous system continues to release epinephrine and norepinephrine to tighten the blood vessels, the heart rate increases further, which may cause shakiness, forceful or skipped heartbeats, and chest pain. Some individuals with POTS develop hypotension with prolonged standing. Other individuals develop an increase in blood pressure when they stand. POTS symptoms sometimes worsen when the subject is in a warm environment, or in a situation involving a lot of standing, or if fluid and salt intake have not been adequate.

In accordance with embodiments, the patient may present with mild and/or infrequent or sporadic symptoms of POTS, or may present with moderate symptoms of POTS, or the patient may present with severe, disabling symptoms.

In various embodiments, the subject may have one or more of neuropathic POTS, hyperadrenergic POTS, hypovolemic POTS, and secondary POTS. Neuropathic POTS is associated with damage to small fiber nerves (small-fiber neuropathy). These nerves regulate the constriction of the blood vessels in the limbs and abdomen. Hyperadrenergic POTS is associated with elevated levels of the stress hormone norepinephrine. Hypovolemic POTS is associated with abnormally low levels of blood (hypovolemia). Secondary POTS is associated with another condition known to potentially cause autonomic neuropathy, such as diabetes, Lyme disease, or autoimmune disorders such as lupus (SLE) or Sjögren's syndrome.

In embodiments, the patient having POTS has an associated condition, such as Ehlers-Danlos syndrome. Ehlers-Danlos syndrome is a group of inherited disorders that affect connective tissues—primarily the skin, joints, and blood vessel walls. A more severe form of the disorder, called vascular Ehlers-Danlos syndrome, can cause the walls of blood vessels, intestine, or uterus to rupture. In some embodiments, the subject has hypermobility spectrum disorders/hypermobile Ehlers-Danlos syndrome (HSD/hED) (e.g., Type III), and who may have an associated functional gastrointestinal disorder. For example, the subject may have gastrointestinal symptoms such as dysphagia, reflux, nausea, abdominal pain, and bloating.

GIP is an insulinotropic peptide released from intestinal K-cells during the postprandial period. As an incretin, GIP stimulates insulin secretion by stimulating pancreatic beta cells in response to food intake. GIP, also notated as GIP(1-42) (SEQ ID NO: 1), primarily circulates as a 42-amino acid polypeptide, but is also present in a form lacking the first 2 N-terminal amino acids (GIP(3-42)). GIP functions via binding to its cognate receptor (GIPR) found on the surface of target cells. GIPR is a member of the glucagon-secretin family of G-protein coupled receptors (GPCRs), possessing seven transmembrane domains. Native GIP(1-42) and derivatives bind to GIPR with high affinity and possess agonist properties. GIP receptors are described, for example, in Pujadas G. and Drucker D J, Vascular Biology of Glucagon Receptor Superfamily Peptides: Mechanistic and Clinical Relevance, Endocrine Reviews, Volume 37, Issue 6, (2016).

In embodiments, administration of the molecular antagonist occurs before or concurrently with consuming a meal, optionally immediately prior to consuming a meal, to prevent or reduce postprandial POTS symptoms. For example, in these embodiments administration of the molecular antagonist occurs no more than about 10 minutes, or no more than about 20 minutes, or no more than about 30 minutes, or no more than about one hour before consuming (e.g., initiating) a meal. In other embodiments, the molecular antagonist is administered during or after consuming a meal, optionally immediately after consuming a meal. In some embodiments, administration of the molecular antagonist occurs within about 15 minutes, within about 30 minutes, or within about one hour after consuming a meal, or otherwise upon the onset of symptoms. In other embodiments, the molecular antagonist is administered on a set frequency, such as once or twice daily, once weekly, once bi-weekly, or once monthly. In some embodiments, the molecular antagonist of GIP is administered parenterally, such as intramuscularly, subcutaneously, intradermally, or intravenously. In certain embodiments, the GIP antagonist is administered by subcutaneous administration.

In various embodiments, the molecular antagonist binds GIP and/or GIP receptor (GIPR). In embodiments, the molecular antagonist is an antibody, an antigen-binding fragment, a peptide, an aptamer, an adnectin, a single-domain antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody, a Transbody, an Anticalin, an AdNectin, an Affilin, a Microbody, a peptide aptamer, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)₂, a peptide mimetic molecule, or a synthetic molecule or as described in U.S. Patent Nos. or Patent Publication Nos. U.S. Pat. No. 7,417,130; US 2004/132094; U.S. Pat. No. 5,831,012; US 2004/023334; U.S. Pat. Nos. 7,250,297; 6,818,418; US 2004/209243; U.S. Pat. Nos. 7,838,629; 7,186,524; 6,004,746; 5,475,096; US 2004/146938; US 2004/157209; U.S. Pat. Nos. 6,994,982; 6,794,144; US 2010/239633; U.S. Pat. No. 7,803,907; US 2010/119446; and/or U.S. Pat. No. 7,166,697, all of which are hereby incorporated by reference in their entireties. See also, Storz MAbs. 2011 May-June; 3(3): 310-317, which is incorporated by reference in its entirety. Exemplary targeting agents include antigen-binding antibody fragments, such as but not limited to F(ab′)₂ or Fab, a single chain antibody, a bi-specific antibody, or a single domain antibody.

Monoclonal antibodies binding to GIP are described, for example, in U.S. Pat. Nos. 9,771,422, 10,196,441, and 10,538,586 (which patents are hereby incorporated by reference in their entireties), and which may be employed in accordance with embodiments of this disclosure. Other antagonists include peptide antagonists of GIPR such as GIP(3-30)NH₂, which can be employed in some embodiments. See Gasbjerg L S, et al., GIP(3-30)NH2 is an efficacious GIP receptor antagonist in humans: a randomised, double-blinded, placebo-controlled, crossover study, Diabetologia volume 61 (2018). Generally, peptide antagonists can be prepared by derivatizing the GIP sequence or fragments thereof, including with one or more non-natural amino acids and/or (d)-amino acids.

In some embodiments, the GIP antagonist has a size of less than about 40 kDa or less than about 30 kDa, and in these embodiments the GIP antagonist will have a short half-life suitable for preventing or inhibiting the onset of symptoms (e.g., postprandial symptoms), while not otherwise impacting the important physiological role of GIP in the absence of symptoms (or after symptoms are controlled). In such embodiments, the GIP antagonist may be a scFv or Fab or maybe a peptide antagonist of GIPR. In other embodiments, the antagonist is a full monoclonal antibody, which is suitable for once daily or once weekly administration, for example, to control GIP levels.

In embodiments, the molecular antagonist is a monoclonal antibody or antigen binding fragment thereof that binds to GIP and neutralizes GIP binding to GIPR. An exemplary monoclonal antibody comprises the Light Chain sequence of SEQ ID NO: 2 and the Heavy Chain Variable Domain of SEQ ID NO: 14. In various embodiments, the monoclonal antibody has a Light Chain sequence that has at least 80%, at least 90%, at least 95%, or at least 98% identity to the amino acid sequence of SEQ ID NO: 2. In various embodiments, the monoclonal antibody has a Heavy Chain Variable Domain that has at least 80%, at least 90%, at least 95%, or at least 98% identity to the amino acid sequence of SEQ ID NO: 14. In such embodiments, the Light Chain sequence may have a CDR1 of SEQ ID NO: 3, a CDR2 of SEQ ID NO: 4, and a CDR3 of SEQ ID NO: 5. Alternatively, each such light chain CDR may have one or two modifications (i.e., amino acid substitutions). In such embodiments, the Heavy Chain Variable Region may have a CDR1 of SEQ ID NO: 15, a CDR2 of SEQ ID NO: 16, and a CDR3 of SEQ ID NO: 17. Alternatively, each such heavy chain CDR may have one or two modifications (i.e., amino acid substitutions).

In certain embodiments, the molecular antagonist is a humanized monoclonal antibody or antigen binding fragment thereof. An exemplary monoclonal antibody comprises the humanized Light Chain sequence of SEQ ID NO: 6 or SEQ ID NO: 7, and/or comprises the humanized Heavy Chain Variable Domain of SEQ ID NO: 18, SEQ ID NO: 19, or SEQ ID NO: 20. In various embodiments, the monoclonal antibody has a humanized Light Chain sequence that has at least 80%, at least 90%, at least 95%, or at least 98% identity to the amino acid sequence of SEQ ID NO: 6 or SEQ ID NO: 7. In various embodiments, the monoclonal antibody has a humanized Heavy Chain Variable Domain that has at least 80%, at least 90%, at least 95%, or at least 98% identity to the amino acid sequence of SEQ ID NO: 18, SEQ ID NO: 19, or SEQ ID NO: 20. In such embodiments, the humanized Light Chain may have a CDR1 of SEQ ID NO: 9; a CDR2 of SEQ ID NO: 10, SEQ ID NO: 12, or SEQ ID NO: 13; and a CDR3 of SEQ ID NO: 11. Alternatively, each such light chain CDR may have one or two modifications (i.e., amino acid substitutions). In such embodiments, the humanized Heavy Chain Variable Domain may have a CDR1 of SEQ ID NO: 15, a CDR2 of SEQ ID NO: 16, and a CDR3 of SEQ ID NO: 17. Alternatively, each such heavy chain CDR may have one or two modifications (i.e., amino acid substitutions).

In some embodiments, the molecular antagonist is a single-chain variable fragment (scFv), an F(ab′)2 fragment, a Fab or Fab′ fragment, a diabody, a triabody, a tetrabody, derived from a monoclonal antibody described herein.

In embodiments, the molecular antagonist is a whole monoclonal antibody comprising human constant regions. In embodiments, the molecular antagonist is a whole monoclonal antibody formed from light chains and heavy chains having the variable regions or domains, combined with human constant regions. The constant region of the heavy chain can be any human isotype, including IgA1, IgA2, IgD, IgE, IgG1, IgG2, IgG3, IgG4, or IgM. The human constant region of the light chain can be the kappa or lambda isotype. In specific embodiments, the heavy chain constant region is the IgG1 isotype, and the light chain constant region is the kappa isotype. In some embodiments, the monoclonal antibody has one or more amino acid substitutions for pharmacodynamic enhancement. Such amino acid modifications include, without limitation, a mutation in the crystallizable fragment (Fc) domain selected from one or more M252Y, S254T, T256E, M428L, and N434S. For example, the monoclonal antibody may have a YTE modification (M252Y, S254T, T256E) for extended half-life.

In some embodiments, the monoclonal antibody antagonist of GIP (whether whole antibody or antigen-binding fragment) has a binding affinity for GIP characterized by an K_{D} of less than about 50 nM, or less than about 20 nM, or less than about 10 nM, or less than about 5 nM, or less than about 1 nM.

In some embodiments, the compositions are formulated as a composition adapted for parenteral administration. Dosage forms suitable for parenteral administration (e.g., intravenous, intramuscular, i.v. infusion, and subcutaneous) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents. In still other embodiments, the one or more unit doses are provided as an injection pen for convenient self-administration.

In some embodiments, the compositions may additionally include pharmaceutically acceptable excipients or carriers. Particular excipients include buffering agents, surfactants, preservative agents, bulking agents, polymers, and stabilizers, which are useful with these molecular antagonists. Buffering agents are used to control the pH of the composition. Surfactants are used to stabilize proteins, inhibit protein aggregation, inhibit protein adsorption to surfaces, and assist in protein refolding. Exemplary surfactants include Tween 80, Tween 20, Brij 35, Triton X-10, Pluronic F127, and sodium dodecyl sulfate. Preservatives are used to prevent microbial growth. Examples of preservatives include benzyl alcohol, m-cresol, and phenol. Bulking agents are used during lyophilization to add bulk. Hydrophilic polymers such as dextran, hydroxyl ethyl starch, polyethylene glycols, and gelatin can be used to stabilize proteins. Polymers with nonpolar moieties such as polyethylene glycol can also be used as surfactants. Protein stabilizers can include polyols, sugars, amino acids, amines, and salts. Suitable sugars include sucrose and trehalose. Amino acids include histidine, arginine, glycine, methionine, proline, lysine, glutamic acid, and mixtures thereof. Proteins like human serum albumin can also competitively adsorb to surfaces and reduce aggregation of the active agent. Particular formulation components can serve multiple purposes. For example, histidine can act as a buffering agent and an antioxidant. Glycine can be used as a buffering agent and as a bulking agent.

In certain aspects and embodiments, the composition comprising a GIP molecular antagonist is contained in an injection pen for convenient self-administration. Injection pens may house, for example, from 1 to 100 unit doses, such as from 10 to 60 unit doses. In some embodiments, the injection pen contains and delivers a unit dose of from about 10 mg to about 500 mg of the molecular antagonist. In some embodiments, the injection pen contains and delivers a unit dose of from about 50 mg to about 200 mg of the molecular antagonist, or from about 10 to about 100 mg of the molecular antagonist. In embodiments, the unit doses are no more than about 1.5 mL, or no more than about 1 mL in volume. In some embodiments, the unit doses are no more than 0.8 mL in volume or no more than about 0.50 mL in volume.

The singular forms “a,” “an,” and “the” include plural referents unless the context clearly dictates otherwise.

As used in the specification and in the claims, the open-ended transitional phrases “comprise(s),” “include(s),” “having,” “contain(s),” and variants thereof require the presence of the named features/steps and permit the presence of other features/steps. These phrases should also be construed as disclosing the closed-ended phrases “consist of” or “consist essentially of” that permit only the named features/steps and unavoidable impurities, and exclude other features/steps.

As used herein, the term “about” means ±10% of a numerical value, unless the context requires otherwise.

The term “identity” refers to the similarity between a pair of sequences (nucleotide or amino acid). Identity is measured by dividing the number of identical residues by the total number of residues and multiplying the product by 100 to achieve a percentage. Thus, two copies of exactly the same sequence have 100% identity, but sequences that are less highly conserved and have deletions, additions, or replacements may have a lower degree of identity. Those skilled in the art will recognize that several computer programs, such as those that employ algorithms such as BLAST, are available for determining sequence identity. BLAST nucleotide searches are performed with the NBLAST program, and BLAST protein searches are performed with the BLASTP program, using the default parameters of the respective programs.

The term CDR refers to a complementarity-determining region. CDRs are part of the variable chains in immunoglobulins (antibodies). A set of CDRs constitutes a paratope.

1. A method for treating postural orthostatic tachycardia syndrome (POTS) in a patient, comprising administering a composition comprising an effective amount of a molecular antagonist of Glucose-dependent Insulinotropic Polypeptide (GIP) to a patient in need thereof.

2. The method of claim 1, wherein the patient presents with mild symptoms of POTS.

3. The method of claim 1, wherein the patient presents with moderate symptoms of POTS.

4. The method of claim 1, wherein the patient has a severe form of POTS, optionally wherein the patient presents disabling symptoms.

5. The method of any one of claims 1 to 4, wherein the patient has one or more of neuropathic POTS, hyperadrenergic POTS, hypovolemic POTS, and secondary POTS.

6. The method of any one of claims 1 to 5, wherein the patient further has hypermobility spectrum disorders/hypermobile Ehlers-Danlos syndrome (HSD/hEDS).

7. The method of any one of claims 1 to 6, wherein the patient exhibits presyncopal symptoms.

8. The method of claim 7, wherein the presyncopal symptoms are selected from lightheadedness, upright tachycardia, mental clouding, blurred vision, shortness of breath, rapid heartbeat, tremulousness, chest discomfort, headache, nausea, and dizziness.

9. The method of any one of claims 1 to 8, wherein the molecular antagonist is administered parenterally.

10. The method of claim 9, wherein the parenteral route of administration is selected from intramuscular administration, subcutaneous administration, intradermal administration, and intravenous (IV) administration.

11. The method of any one of claims 1 to 10, wherein the administration occurs before consuming a meal, such as no more than about one hour before a meal.

12. The method of any one of claims 1 to 10, wherein the administration occurs during or after consuming a meal.

13. The method of claim 12, wherein the administration occurs within about an hour after consuming a meal.

14. The method of any one of claims 1 to 10, wherein the administration is upon the onset of symptoms.

15. The method of any one of claims 1 to 10, wherein administration is about once or twice daily.

16. The method of any one of claims 1 to 10, wherein administration is about once per week.

17. The method of any one of claims 1 to 10, wherein administration is about once every other week.

18. The method of any one of claims 1 to 10, wherein administration is about once per month.

19. The method of any one of claims 1 to 18, wherein the molecular antagonist binds GIP or GIP receptor (GIPR).

20. The method of claim 19, wherein the molecular antagonist is a peptide antagonist of GIP receptor.

21. The method of claims 19, wherein the molecular antagonist is a monoclonal antibody or antigen-binding portion thereof that binds and neutralizes GIP.

22. The method of claim 21, wherein the molecular antagonist is a single-chain variable fragment (scFv), or a Fab or Fab′ fragment.

23. The method of claim 21, wherein the molecular antagonist is a monoclonal antibody comprising a Light Chain amino acid sequence of SEQ ID NO: 2 or a derivative thereof, and a Heavy Chain Variable Domain of SEQ ID NO: 14 or a derivative thereof.

24. The method of claim 23, wherein the monoclonal antibody comprises a Light Chain amino acid sequence that has at least 80%, at least 90%, at least 95%, or at least 98% identity to the amino acid sequence of SEQ ID NO: 2.

25. The method of claim 24, wherein the Light Chain has a CDR1 of SEQ ID NO: 3, a CDR2 of SEQ ID NO: 4, and a CDR3 of SEQ ID NO: 5, wherein each light chain CDR may optionally have one or two amino acid substitutions with respect to SEQ ID NO: 3, SEQ ID NO: 4, or SEQ ID NO: 5.

26. The method of any one of claims 23 to 25, wherein the monoclonal antibody comprises a Heavy Chain Variable Domain that has at least 80%, at least 90%, at least 95%, or at least 98% identity to the amino acid sequence of SEQ ID NO: 14.

27. The method of claim 26, wherein the Heavy Chain Variable Domain has a CDR1 of SEQ ID NO: 15, a CDR2 of SEQ ID NO: 16, and a CDR3 of SEQ ID NO: 17, wherein each such heavy chain CDR may optionally have one or two modifications amino acid substitutions with respect to SEQ ID NO: 15, SEQ ID NO: 16, or SEQ ID NO: 17.

28. The method of claim 21, wherein the molecular antagonist is a humanized antibody.

29. The method of claim 28, wherein the humanized monoclonal antibody comprises the Light Chain amino acid sequence of SEQ ID NO: 6 or SEQ ID NO: 7, or a derivative thereof, and/or comprises the Heavy Chain Variable Domain of SEQ ID NO: 18, SEQ ID NO: 19, or SEQ ID NO: 20, or a derivative thereof.

30. The method of claim 29, wherein the monoclonal antibody comprises a humanized Light Chain amino acid sequence that has at least 80%, at least 90%, at least 95%, or at least 98% identity to the amino acid sequence of SEQ ID NO: 6 or SEQ ID NO: 7.

31. The method of claim 30, wherein the humanized Light Chain has a CDR1 of SEQ ID NO: 9; a CDR2 of SEQ ID NO: 10, SEQ ID NO: 12, or SEQ ID NO: 13; and a CDR3 of SEQ ID NO: 11, wherein each such light chain CDR may optionally have one or two amino acid substitutions with respect to SEQ ID NO: 9, SEQ ID NO: 10, SEQ ID NO: 12, SEQ ID NO: 13, or SEQ ID NO: 11.

32. The method of any one of claims 29 to 31, wherein the monoclonal antibody comprises a humanized Heavy Chain Variable Domain that has at least 80%, at least 90%, at least 95%, or at least 98% identity to the amino acid sequence of SEQ ID NO: 18, SEQ ID NO: 19, or SEQ ID NO: 20.

33. The method of claim 32, wherein the humanized Heavy Chain Variable Domain has a CDR1 of SEQ ID NO: 15, a CDR2 of SEQ ID NO: 16, and a CDR3 of SEQ ID NO: 17, wherein each such heavy chain CDR may optionally have one or two amino acid substitutions with respect to SEQ ID NO: 15, SEQ ID NO: 16, or SEQ ID NO: 17.

34. The method of any one of claims 21 to 33, wherein the molecular antagonist has a binding affinity for GIP characterized by a K_{D} of less than about 50 nM, or less than about 20 nM, or less than about 10 nM, or less than about 5 nM, or less than about 1 nM.

35. The method of any one of claims 1 to 34, wherein the composition is contained in an injection pen.

36. The method of claim 35, wherein the injection pen contains and delivers a unit dose of from about 10 mg to about 500 mg of the molecular antagonist.

37. The method of claim 36, wherein the injection pen contains and delivers a unit dose of from about 50 mg to about 200 mg of the molecular antagonist.

38. The method of any one of claims 35 to 37, wherein the unit doses are no more than about 1.5 mL, or no more than about 1 mL in volume.

39. The method of any one of claims 1 to 38, wherein administration results in decreased plasma levels of GIP.

40. The method of any one of claims 1 to 39, wherein administration results in reduced chronic presyncopal symptoms selected from one or more of lightheadedness, upright tachycardia, mental clouding, blurred vision, shortness of breath, rapid heartbeat, tremulousness, chest discomfort, headache, nausea, and dizziness.

41. The method of any one of claims 1 to 40, wherein administration results in modulation of and/or increased upright stroke volume.

42. The method of any one of claims 1 to 41, wherein administration results in reduced upright norepinephrine and/or epinephrine levels.

43. An injection pen comprising a composition comprising an effective amount of a molecular antagonist of Glucose-dependent Insulinotropic Polypeptide (GIP) as described herein.


SEQUENCE LISTING (23 sequences)

SEQ ID NO: 1 - protein, 42 aa, synthetic construct
YAEGTFISDYSIAMDKIHQQDFVNWLLAQKGKKNDWKHNITQ

SEQ ID NO: 2 - protein, 111 aa, synthetic construct
DIQLTQSPSTMAASPGEKITITCSASSSISSNSLHWYQQKPGFSPKLLIYRTSNLASGVPGRFSGSGSGTSYSLTIGTMETEDVATYYCQQGSSFPRMLTFGTGTKLEIKR

SEQ ID NO: 3 - protein, 12 aa, synthetic construct
SASSSISSNSLH

SEQ ID NO: 4 - protein, 7 aa, synthetic construct
RTSNLAS

SEQ ID NO: 5 - protein, 11 aa, synthetic construct
QQGSSFPRMLT

SEQ ID NO: 6 - protein, 111 aa, synthetic construct
DIQMTQSPSSVSASVGDRVTITCRASSSISSNSLHWYQQKPGKAPKLLIYRTSSLQSGVPSRFSGSGSGTDYTLTISSLQPEDFATYYCQQGSSFPRMLTFGQGTKLEIKR

SEQ ID NO: 7 - protein, 111 aa, synthetic construct
DIQLTQSPSSVSASVGDRVTITCRASSSISSNSLHWYQQKPGKAPKLLIYRTSNLQSGVPSRFSGSGSGTDYTLTISSLQPEDFATYYCQQGSSFPRMLTFGQGTKLEIKR

SEQ ID NO: 8 - protein, 111 aa, synthetic construct
DIQMTQSPGTLSLSPGERATLSCRASSSISSNSLHWYQQKPGQAPRLLIYRTSNRATGIPARFSGSGSGTDYTLTISSLEPEDFAVYYCQQGSSFPRMLTFGGGTKVEIKR

SEQ ID NO: 9 - protein, 12 aa, synthetic construct
RASSSISSNSLH

SEQ ID NO: 10 - protein, 7 aa, synthetic construct
RTSNLQS

SEQ ID NO: 11 - protein, 11 aa, synthetic construct
QQGSSFPRMLT

SEQ ID NO: 12 - protein, 7 aa, synthetic construct
RTSSLQS

SEQ ID NO: 13 - protein, 7 aa, synthetic construct
RTSNRAT

SEQ ID NO: 14 - protein, 117 aa, synthetic construct
QVQLQESGAELVRSGASVKLSCTASGFNIRDYYLHWIKQRPEQGLEWIGWIDPENGDTEYAPKFQDKATVTADTSSNTAYLQLNSLTSEDTAVYYCNVYGIYFMDYWGQGTTVTVSS

SEQ ID NO: 15 - protein, 10 aa, synthetic construct
GFNIRDYYLH

SEQ ID NO: 16 - protein, 7 aa, synthetic construct
APKFQDK

SEQ ID NO: 17 - protein, 10 aa, synthetic construct
NVYGIYFMDY

SEQ ID NO: 18 - protein, 117 aa, synthetic construct
QVQLVQSGAEVKKPGATVKISCKVSGFNIRDYYLHWVQQAPGKGLEWMGWIDPENGDTEYAPKFQGRVTITADTSTDTAYMELSSLRSEDTAVYYCNVYGIYFMDYWGQGTMVTVSS

SEQ ID NO: 19 - protein, 117 aa, synthetic construct
QVQLVQSGAEVKKPGATVKISCKASGFNIRDYYLHWVQQAPGKGLEWMGWIDPENGDTEYAPKFQGRVTITADTSTNTAYMELSSLRSEDTAVYYCNVYGIYFMDYWGQGTMVTVSS

SEQ ID NO: 20 - protein, 117 aa, synthetic construct
QVQLQESGAEVKKPGASVKVSCKASGFNIRDYYLHWVRQAPGQGLEWMGWIDPENGDTEYAPKFQGRVTTTADTSISTAYMELSRLRSDDTAVYYCNVYGIYFMDYWGQGTLVTVSS

SEQ ID NO: 21 - protein, 10 aa, synthetic construct
GFNIRDYYLH

SEQ ID NO: 22 - protein, 7 aa, synthetic construct
APKFQGR

SEQ ID NO: 23 - protein, 10 aa, synthetic construct
NVYGIYFMDY